(19) United States
(12) Patent Application Publication
(10) Pub. No.: US 2009/0200393 A1
(43) Pub. Date: Aug. 13, 2009

(54) METHOD AND APPARATUS FOR DIFFUSING THE FRAGRANCE OF A BURNING CANDLE

(76) Inventor: Victor Avelar, Lincoln, RI (US)

Correspondence Address: WOLF GREENFIELD & SACKS, P.C., 600 ATLANTIC AVENUE, BOSTON, MA, 02210-2206, US

(21) Appl. No.: 12/363,843

(22) Filed: Feb. 2, 2009

Related U.S. Application Data
(60) Provisional application No. 61/024,943, filed on Jan. 31, 2008.

(51) Int. Cl. A61L 9/04

(52) U.S. Cl. 239/60

(57) ABSTRACT

A method and apparatus for controlling a candle flame. A candle flame may be provided in a controllable environment, e.g., a semi-enclosed area formed at least in part by a cover. Air may be forced into the semi-enclosed area by an air movement device (e.g., a fan) to diffuse a candle's fragrance. The fragrance diffusion rate may be reduced by reducing (e.g., stopping) air flow into the semi-enclosed area.

### BACKGROUND OF INVENTION

This invention relates to diffusing the fragrance of a burning candle.

Burning a stand-alone scented candle does not quickly fill a room with fragrance. Typically, the air flows in the room do not have enough turbulence and velocity to pick up the fragrance from a burning candle and diffuse them throughout the room in a timely manner (i.e. less than 30 minutes).

### SUMMARY OF INVENTION

Aspects of the invention provide methods and apparatus for diffusing the fragrance of a burning candle. As used herein, a “candle” refers to any device or substance that may have an open flame or otherwise be burned, including stick candles, votive candles, jar candles, incense, oil lamps or any other device having a wick to burn a fuel, etc. In some aspects of the invention, a candle may be burned in a semi-enclosed area so that air turbulence is created within. Air flow into the semi-enclosed area may be controlled so as to increase or decrease the rate of fragrance diffusion. For example, a fan may move air into or out of the semi-enclosed area, creating air turbulence.

In one aspect of the invention, a fragrance diffusion system for a candle includes a cover that defines a semi-enclosed area arranged to house a candle having a burning flame for an extended period. The burning flame may be positioned above a lowermost portion of the bottom of the cover, which may have one or more openings in portions above the bottom. The cover may have an opening at the top or bottom to allow air to enter the semi-enclosed area. An air supply may provide air into or out of the semi-enclosed area, thereby diffusing the fragrance of a burning candle into a room.

In one aspect of the invention, a candle fragrance diffusion system includes an enclosure that defines a semi-enclosed area in which a burning flame of a candle is positionable for an extended period, such as 1 hour or more. In one embodiment the burning flame may be positioned in the semi-enclosed area approximately at a vertical axis. A support may be constructed and arranged to support the candle with the burning flame positioned in the semi-enclosed area, and an air inlet may direct air into the semi-enclosed area from at least one location above the burning flame, e.g., the enclosure may include one or more openings located above the burning flame through which air enters the semi-enclosed area. An exhaust outlet may direct exhaust from the semi-enclosed area in at least two different radial directions relative to the vertical axis, and an air movement device may cause air to be pulled into the semi-enclosed area through the air inlet and cause fragranced air from the burning candle to be exhausted from the semi-enclosed area through the exhaust outlet.

In one embodiment, air flow through the enclosure may be generally opposite to that which would be induced by standard thermal convection, i.e., air may enter the enclosure at the top and flow downwardly into the semi-enclosed area. Exhaust from the burning flame may exit the enclosure at the bottom of the enclosure, e.g., to flow outwardly in radial directions relative to the vertical axis.

In another aspect of the invention, a candle fragrance diffusion system includes an enclosure that defines a semi-enclosed area in which a burning flame of a candle is positionable for an extended period with the burning flame being positioned in the semi-enclosed area approximately at a vertical axis. A support may be constructed and arranged to support the candle with the burning flame positioned in the semi-enclosed area, and an air inlet may direct air into the semi-enclosed area. An air movement device may cause air to be provided into the semi-enclosed area and cause fragranced air from the burning candle to be exhausted from the semi-enclosed area in a vertically upward direction, e.g., air may flow upwardly and exit via an exhaust outlet at a top of the enclosure. In one embodiment, the air inlet is constructed and arranged to introduce air into the semi-enclosed area so that the burning flame remains generally aligned along the vertical axis while burning. This arrangement may help a candle to burn more evenly, e.g., to help prevent dripping of the unburned candle wax.

In another aspect of the invention, a candle fragrance diffusion system includes an enclosure that defines a semi-enclosed area in which a burning flame of a candle is positionable for an extended period with the burning flame being positioned in the semi-enclosed area approximately at a vertical axis. The enclosure may include an opening near a top of the enclosure and an opening near the bottom of the enclosure. A support may be constructed and arranged to support the candle with the burning flame positioned in the semi-enclosed area, and exhaust outlets may direct exhaust from the semi-enclosed area away from the vertical axis. An air movement device may cause air to be pulled into the semi-enclosed area through the opening at the top of the enclosure and cause fragranced air from the burning candle to be exhausted from the semi-enclosed area through the opening near the bottom of the enclosure.

These and other aspects of the invention will be apparent and/or obvious from the following description and appended claims.

### BRIEF DESCRIPTION OF THE DRAWINGS

Aspects of the invention are described with reference to the following drawings in which like numerals reference like elements, and wherein:

FIG. 1 is a perspective view of an illustrative embodiment of a candle fragrance diffusion system in accordance with the invention in which with combustion products are exhausted radially outward;

FIG. 2 shows a side view of the FIG. 1 embodiment;

FIG. 3 shows an exploded view of the FIG. 1 embodiment;

FIG. 4 shows a perspective view of a candle fragrance diffusion system for use with a candle in which combustion products are exhausted upwardly; and

FIG. 5 shows a side view of the FIG. 4 embodiment.

### DETAILED DESCRIPTION

Various aspects of the invention are described below with reference to illustrative embodiments. However, it should be understood that aspects of the invention are not limited to those embodiments described below, but instead may be used in any suitable system or arrangement.

Aspects of the invention provide a controllable environment, e.g., a semi-enclosed area, in which the candle fragrance diffusion may be controlled. Thus, the candle flame may be housed in a cover or other structure and have its fragrance diffusion rate adjusted. The candle fragrance diffusion rate may be adjusted by controlling air flow into the controllable environment.

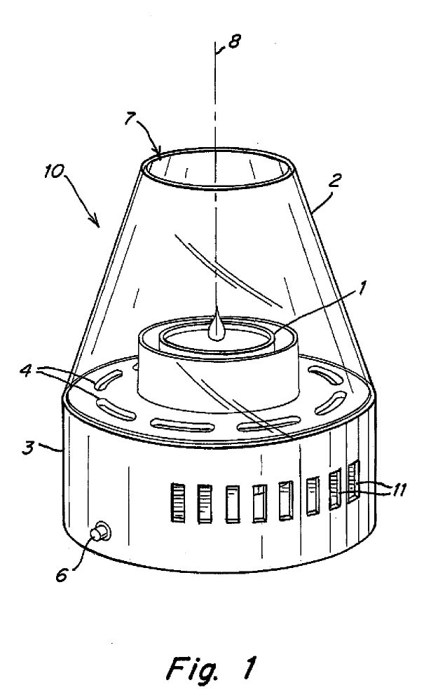

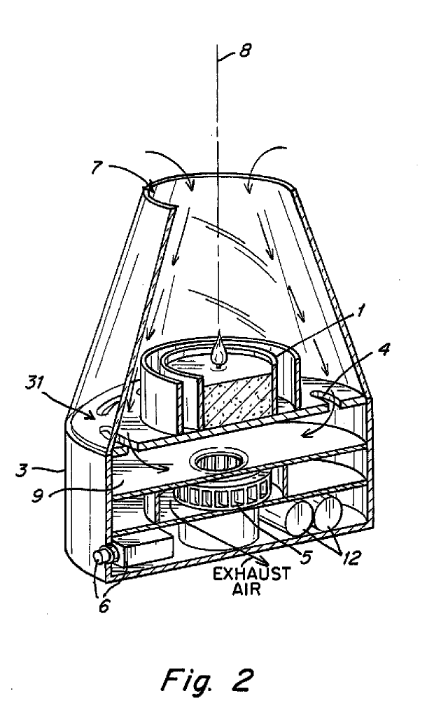

FIG. 1 shows an illustrative embodiment of a candle fragrance diffusion system 10 that incorporates various aspects of the invention. In this illustrative embodiment, a candle 1 is arranged within a cover 2 that defines a semi-enclosed area in which the candle's flame may burn for an extended period, e.g., 1 minute, 10 minutes, 1 hour, or more. Although the cover 2 may be made in any suitable arrangement, in this embodiment the cover 2 is made of glass and has a conical shape. The cover 2 may have one or more holes or openings to allow air to pass, above its bottom portion. The cover 2 may also have one or more holes or openings to allow air to pass above its top portion. In one illustrative embodiment, the cover 2 defines an interior area having a volume slightly larger than the enclosed candle with the bottom of the cover 2 having a diameter approximately 1 inch larger than the candle diameter. The candle 1, which may be a tealight candle, may be placed inside the semi-enclosed area of the cover 2 on a support 3 that has at least one opening 4 that allows air to be drawn in from the cover's top opening 7. As illustrated in FIG. 2, an air movement device 5, e.g., a fan, draws in air from opening 7 (part of an air inlet) through opening(s) 4 and into an air chamber 9 (part of an exhaust outlet). The support 3 and one or more openings 4 are arranged so that the candle's fragrance diffusion is adjusted if the air movement device 5 increases or decreases the intake of air through the openings 4 and into the air chamber 9. That is, the fan 5 moves air through the openings 4 at a rate to provide sufficient air to exhaust combustion products (e.g. a candle's fragrance) through the exhaust vents 11 in FIG. 1. In one embodiment, the support 3 has a plurality of openings 4 around the circumference in at least partial registration with the opening at the bottom of the cover 2. Each of the openings 4 may have a size of about 0.1 sq. inch and be equally spaced from each other about ¼ inch apart. In the illustrative embodiment, the fan has a diameter of about 1.5 inches, is run by a motor rated at about 2 watts, and is spaced at about ½ to 1 inch below the openings 4.

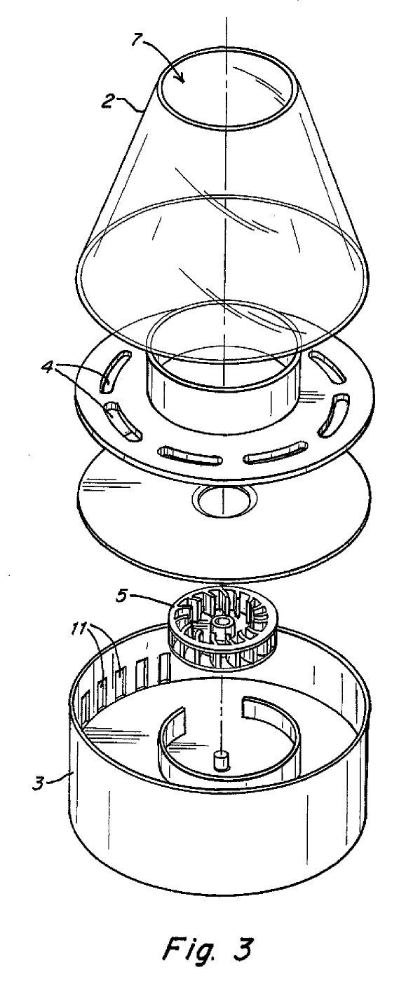

FIG. 2 shows a cross-sectional side view of the FIG. 1 embodiment and depicts air flow into the semi-enclosed area defined by the cover 2. The air is drawn from opening 7, into openings 4, and into air chamber 9. The air is then drawn into the fan 5 and exhausted radially out through exhaust vents 11. FIG. 3 provides a perspective of the fan housing and the exhaust vents 11. In this embodiment and as shown in FIG. 2, the fan 5 causes air to be drawn downward from cover's 2 top opening 7 and toward a plurality of exhaust chamber inlet ducts 4, which each direct the air to move in a equally distributed direction into the air chamber 9 relative to a vertical axis 8 at which the candle flame is approximately located. As can be seen in FIG. 2, the air entering via the inlet openings 4 is directed downward and radially inward into the fan 5. The exhaust may contact the cover 2 and create a turbulent environment in the semi-enclosed area. By introducing air into the chamber 9 via the openings 4 in two or more directions, the flame may burn in a more balanced way, thereby avoiding “push” of the flame (causing the flame to lean in a direction away from vertical), avoiding substantial leaning of the flame and/or other deviation of the flame from a direction aligned with the vertical axis 8. As a result, the flame may burn the candle more evenly, creating less smoke and/or avoiding damage to the candle holder (i.e., a cup or other container in which the candle wax and wick are provided).

As shown in FIG. 2, exhaust fragrance produced by the candle 1 exits the semi-enclosed area via exhaust vents 11 on the side of support 3. Pressure created by the fan forces the exhaust fragrances through the exhaust vents 11. As discussed above, forced exhaust output helps to disperse the fragrance or other materials produced by the candle 1.

Although the inlet openings 4 may be arranged in any suitable way, in this illustrative embodiment, the openings 4 are arranged around a central region 31 of the support 3 as can be seen in FIG. 2. Such an arrangement may aid in proper introduction of air into the semi-enclosed area, mixing of gasses and or fragrance in the semi-enclosed area or other features. Other arrangements are possible, such as having inlet openings 4 arranged in and/or near the cover 2 so that air is removed from the semi-enclosed area in a radial direction toward the vertical axis 8. Similarly, the cover's top opening 7 may be arranged along the sides of the cover 2. Similarly, although in this embodiment the inlet openings 4 are arranged below the candle flame, one or more of the openings may be arranged above the candle flame.

A controller 6 may be used to control the operation of the fan 5 and/or perform other functions. For example, the controller 6 may include one or more switches to control the power provided to the fan 5, e.g., a toggle switch that may be operated by a user to turn the fan on and off. In other embodiments, the controller 6 may control the operation of the fan 5 based on detected environmental conditions or any other suitable criteria. For example, the controller 6 may have a timer that a user may set for a desired period, e.g., 1 hour. The controller 6 may then allow the fan 5 to run for one hour, at which time the fan 5 may be turned off and the candle's fragrance diffusion rate will decrease to a natural rate.

The controller 6 may control air flow into the semi-enclosed space by controlling the fan speed, by adjusting the number or size of openings into the semi-enclosed space or otherwise adjusting the amount of air provided into the semi-enclosed space. When controlling the operation of the fan, the controller 6 need not be only capable of stopping the fan, but rather may adjust the fan speed, e.g., slow the fan sufficiently to slow the candle's fragrance diffusion rate. One or more openings into the semi-enclosed space may be opened or closed or adjusted in size in any suitable way, such as by a movable damper element, one or more flow restrictors, etc. Adjustments to fan speed and/or opening size may be made by a user or by the controller 6. For example, the controller 6 may have an adjustment knob that may be used by an operator to adjust the fan speed, and thus the diffusion rate of the candle's fragrance.

The controller 6 may also provide a visual and/or aural warning based on detected conditions or other system status information. For example, the controller 6 may display a flashing light or emit a warning tone when a high temperature condition exists, the candle flame becomes too high, etc.

The controller 6 may also include a remote control unit by which a user can provide operating instructions to the controller 6 from a distance. A user may use the remote control to adjust the fragrance diffusion rate, turn the fan off, or even cause the controller 6 to automatically light the candle flame (where the controller 6 is suitably equipped). Such a remote control unit may be capable of communicating with multiple candle fragrance diffusion systems 10, e.g., so a user could simultaneously adjust the candle fragrance diffuser for multiple units, or may be capable of individually communicating with one or more candle fragrance diffusion systems 10.

As is described in more detail below, the candle fragrance diffusion system 10 may be arranged in many different ways while achieving desired candle fragrance diffusion control. In the illustrative embodiment of FIG. 1, the cover 2 is made of a translucent glass material that semi-enclosed, i.e., includes at least one hole or other openings other than an opening at the bottom of the cover 2. However, the cover 2 (as well as the support 3) may be made of any suitable material, such as a heat resistant plastic, ceramic or metal, may be transparent, translucent and/or opaque in all or some portions, and/or may have one or more openings in the portion above the cover bottom (e.g., to allow the light and/or scent of a burning candle to escape). It should also be appreciated that the cover 2 may be sized and/or shaped to accommodate any type of candle, such as jar candles, votive candles, stick candles, oil lamps, etc. Moreover, a candle holder, oil lamp reservoir, and/or wick support may be incorporated into the structure of the cover 2. For example, the cover 2 may be made of glass with an integral oil lamp reservoir and wick support molded into the cover 2.

The support 3 in this embodiment has a cylindrical shape, but may be arranged in any suitable way. For example, the support 3 may have a cylindrical shape with a diameter similar to that of the cover 2 (assuming the cover 2 has a cylindrical bottom portion as in FIG. 1). The support 3 may define a space to house the controller, batteries 12, air movement device and/or other components as desired. Also, the support 3 may be arranged to allow air flow into/out of a space defined by the support 3, e.g., to allow air to enter the semi-enclosed space and allow combustion products to escape the space.

The cover 2 may include portions that have an optical effect on the candle light, such as coloring the light (e.g., by filtering), intensity enhancement (e.g., by using materials on the cover that emit light in response to being heated and/or illuminated by the candle flame), focusing or dispersing light (e.g., by using a Fresnel or other lens element or “frosting” portions of the cover 2, etc.).

The cover 2 may optionally include a well or other structure to support a substance to be heated, such as an incense, oil or other odor-producing material, either inside the semi-enclosed space or on the cover exterior. The heat of the candle flame may heat the cover 2, which in turn may heat the substance. For example, the cover 2 in the FIG. 1 embodiment may have a depression or well formed in its top surface to hold an amount of incense. The incense may be heated by the candle flame, and give off a desired odor.

Although not shown in the FIG. 1 embodiment, the cover 2 may be selectively fixed to the support 3, e.g., so that a user cannot lift the cover 2 to expose the candle 1 without unlocking the cover 2 from the support 3. Cover 2 may be secured to the support 3 in any suitable way, such as by one or more clips, a screw-thread connection where a threaded portion of the cover at the bottom engages with a corresponding threaded portion on the support, by positioning a locking cage or other structure over the cover, etc. Also, although the FIG. 1 embodiment has the cover 2 resting on the support 3, the cover 2 may be hingedly engaged with the support so that the cover 2 can be “flipped” up to allow access to the candle 1 and “flipped” down to cover the candle. Other arrangements will be appreciated by those of skill in the art.

One potential advantage of a candle fragrance diffusion system 10 having an air movement device, such as a fan, is that products from the combustion of the candle can be more readily spread throughout a room or space in which the system 10 is located. This may be desired, particularly where the candle gives off a fragrance or other odor. Exhaust gases from the semi-enclosed area of the cover 2 (carrying the desired fragrance or odor) may exit through the one or more openings 11 and/or through other openings in the support 3.

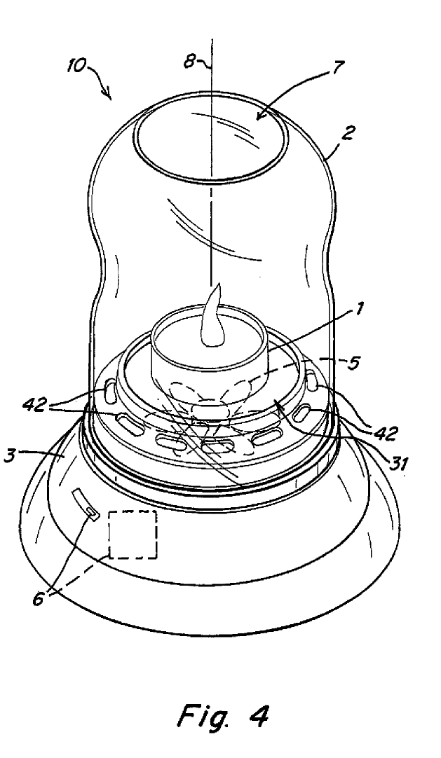

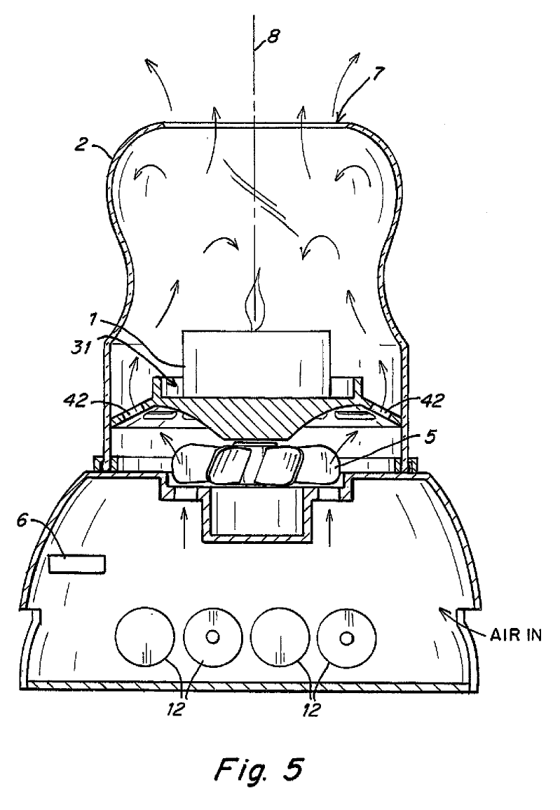

FIGS. 4-5 show another illustrative embodiment of candle fragrance diffusion systems 10 that incorporate various aspects of the invention. FIG. 4 shows an embodiment in which a candle 1 is placed in a cover 2 with opening 7. In this embodiment, as shown in FIG. 5, the air is pulled from below the cover 2 and blown upwards and out through opening 7. Although in this embodiment the cover 2 has a cylindrical shape, it should be understood that the cover 2 may have any suitable shape, such as a square, rectangular, oval, semi-circular or other cross section. A controller 6 including an air movement device moves air into the bottom end of the semi-enclosed area defined by the cover 2. The air movement device causes air to be provided into the semi-enclosed area through supply vents 42. Air is exhausted from the semi-enclosed area through one or more openings 7 at the top of cover 2. The openings 42 may be part of a flow control member that controls the flow rate of air into the semi-enclosed area. As in other embodiments, the size, shape, location or other features of the openings may be adjusted by a user and/or by the controller 6.

Also, the air movement device may be a fan, or any other suitable device, such as an electrostatic air movement device that moves air by accelerating charged air molecules between two oppositely charged electrodes. Such an air movement device may provide air into the semi-enclosed area in a noiseless manner. Other air movement devices, such as a compressed air supply that releases pressurized air into the semi-enclosed space, an air pump (e.g., like that used in fish aquariums), compressor, etc. may be used in this or any of the other embodiments.

The controller 6 and/or air movement device may be powered by standard household electricity, batteries, solar power, etc. The controller 6 may include any suitable electronic circuitry or other devices or components to perform desired functions. For examples the controller 6 may include a programmed microprocessor or other general purpose data processing device, user input/output devices, a visual display, speaker for producing audible signals or tones, a power supply, etc.

While aspects of the invention have been described with reference to illustrative embodiments, aspects of the invention are not limited to those embodiments described. Thus, the description herein of embodiments is intended to be illustrative, not limiting. Aspects of the invention may be used in any suitable arrangement.

1. A candle fragrance diffusion system comprising:
an enclosure that defines a semi-enclosed area in which a burning flame of a candle is positionable for an extended period, the burning flame being positioned in the semi-enclosed area approximately at a vertical axis;
a support constructed and arranged to support the candle with the burning flame positioned in the semi-enclosed area;
an air inlet that direct air into the semi-enclosed area from at least one location above the burning flame;
an exhaust outlet that direct exhaust from the semi-enclosed area in at least two different radial directions relative to the vertical axis; and
an air movement device that causes air to be pulled into the semi-enclosed area through the air inlet and causes fragranced air from the burning candle to be exhausted from the semi-enclosed area through the exhaust outlet;
wherein the enclosure and the semi-enclosed area are arranged so that the fragrance of the candle continues to diffuse while the air movement device pulls air into the semi-enclosed area, and when the air movement device reduces a flow of air pulled into the semi-enclosed area the fragrance diffusion from a scented candle is reduced.

2. The system of claim 1, wherein the exhaust outlet includes a plurality of openings that are equally spaced radially relative to the vertical axis.

3. The system of claim 1, wherein the exhaust outlet directs the exhaust air to move axially downward and then horizontally outwards through exhaust openings in the support in a generally horizontal direction.

4. The system of claim 1, wherein the support includes a central region that supports the candle and at least a portion of the exhaust outlet is formed in the support.

5. The system of claim 4, wherein the support includes a plurality of openings through which exhaust is introduced into an air chamber.

6. The system of claim 4, wherein the openings are located below the burning flame.

7. The system of claim 4, wherein the air movement device draws in exhaust from the air chamber.

8. The system of claim 7, wherein the air movement device directs exhaust to move through openings of the exhaust outlet.

9. The system of claim 8, wherein the air movement device includes a fan located below the central region.

10. The system of claim 9, wherein the enclosure includes a conical translucent member.
wherein the air inlet is constructed and arranged to direct air into the semi-enclosed area so that the burning flame remains generally aligned along the vertical axis while burning.

11. A candle fragrance diffusion system comprising:
an enclosure that defines a semi-enclosed area in which a burning flame of a candle is positionable for an extended period, the burning flame being positioned in the semi-enclosed area approximately at a vertical axis;
a support constructed and arranged to support the candle with the burning flame positioned in the semi-enclosed area;
an air inlet that direct air into the semi-enclosed area;
an exhaust outlet that directs exhaust from the semi-enclosed area in at least one axial direction relative to the vertical axis; and
an air movement device that causes air to be provided into the semi-enclosed area and causes fragranced air from the burning candle to be exhausted from the semi-enclosed area in a vertically upward direction; and
wherein the enclosure and the semi-enclosed area are arranged so that the fragrance of the candle continues to diffuse while the air movement device forces air to move into the semi-enclosed area, and when the air movement device reduces a flow of air provided into the semi-enclosed area the fragrance diffusion from a scented candle is reduced; and
wherein the air inlet is constructed and arranged to introduce air into the semi-enclosed area so that the burning flame remains generally aligned along the vertical axis while burning.

12. The system of claim 11, wherein the air inlet includes a plurality of supply inlets that are equally spaced radially relative to the vertical axis.

13. The system of claim 12, wherein the supply inlets cause supply air to move upwards through at least one exhaust outlet opening at a top of the semi-enclosed cover in a generally vertical direction.

14. The system of claim 12, wherein the support includes a central region that supports the candle and the supply inlets are at least partially formed by the support.

15. The system of claim 14, wherein the support includes a plurality of inlet openings arranged around the central region that form at least part of the supply inlets by which air is introduced into the semi-enclosed area.

16. The system of claim 15, wherein the inlet openings are located below the burning flame.

17. The system of claim 14, wherein the inlet openings are arranged around the central region.

18. The system of claim 13, wherein air introduced into the semi-enclosed area via the plurality of supply inlets reflects off of the sides of the enclosure and is directed generally upward toward the vertical axis.

19. The system of claim 11, wherein the air movement device includes a fan that forces air into the semi-enclosed area via the air inlet.

20. The system of claim 11, wherein the enclosure includes a hemispherical translucent member.

21. The system of claim 11, wherein the exhaust outlet exhausts gases from the semi-enclosed area at a position located above the burning flame.

22. A candle fragrance diffusion system comprising:
an enclosure that defines a semi-enclosed area in which a burning flame of a candle is positionable for an extended period, the burning flame being positioned in the semi-enclosed area approximately at a vertical axis, the enclosure including an opening near a top of the enclosure and an opening near the bottom of the enclosure;
a support constructed and arranged to support the candle with the burning flame positioned in the semi-enclosed area;
exhaust outlets that direct exhaust from the semi-enclosed area away from the vertical axis; and
an air movement device that causes air to be pulled into the semi-enclosed area through the opening at the top of the enclosure and causes fragranced air from the burning candle to be exhausted from the semi-enclosed area through the opening near the bottom of the enclosure; and
wherein the enclosure and the semi-enclosed area are arranged so that the fragrance of the candle continues to diffuse while the air movement device pulls air into the semi-enclosed area, and when the air movement device reduces a flow of air pulled into the semi-enclosed area the fragrance diffusion from a scented candle is reduced.
